SEQUENCE LISTING (6 sequences)

SEQ ID NO: 1 - DNA, 34 bp, Artificial Sequence
  Forward primer for H1N1 NS1 gene
gggcggccgcggatccatggacccaaacactgtg

SEQ ID NO: 2 - DNA, 26 bp, Artificial Sequence
  Reverse primer for H1N1 NS1 gene
caactagtatttcgctttcagtatga

SEQ ID NO: 3 - DNA, 400 bp, Artificial Sequence
  5mNS1 + siRNA NS-128
atggatccaaacactgtgtcaagctttcaggtagattgctttctttggcatgtccgcaaaagagttgcagaccaagaactaggtgatgccccattccttgatcggcttcgccgagatcagaagtccctaagaggaagaggcagcactctcggtctggacatcgaaacagccacccgtgctggaaagcaaatagtggagcggattctgaaggaagaatctgatgaggcactcaaaatgaccatggcctctgtacctgcatcgcgctacctaactgacatgactcttgaggaaatgtcaaggcactggttcatgctcatgcccaagcagaaagtggcaggccctctttgtatcagaatggaccaggcgatcatggataagaacatcatactgaaagcgaaat

SEQ ID NO: 4 - DNA, 13 bp, Artificial Sequence
  Upper strand NotI/XbaI adaptor
ggccgagttgtta

SEQ ID NO: 5 - DNA, 13 bp, Artificial Sequence
  Lower strand NotI/XbaI adaptor
ctagtaacaactc

SEQ ID NO: 6 - DNA, 21 bp, Artificial Sequence
  siRNA oligo NS-128
cggcuucgccgagaucagaat

(19) United States
(12) Patent Application Publication
(10) Pub. No.: US 2006/0282921 A1
(43) Pub. Date: Dec. 14, 2006

(54) Transgenic plant-derived siRNAs for suppression of influenza virus propagation in mammalian cells

(76) Inventors: Eric Lam, Monroe, NJ (US); Lit Man Poon, Belvedere Garden (HK); Yuanxiang Zhou, Hong Kong (HK); Mee Len Chye, Pokfulam (HK); Joseph S.M. Peiris, Hong Kong (HK)

Correspondence Address: COOPER & DUNHAM, LLP, 1185 AVENUE OF THE AMERICAS, NEW YORK, NY, 10036, US

(21) Appl. No.: 11/402,181

(22) Filed: Apr. 10, 2006

Related U.S. Application Data
(60) Provisional application No. 60/673,100, filed on Apr. 20, 2005.

(51) Int. Cl. A01H 1/00; C07H 21/02; A61K 48/00; C12N 5/04; C12N 5/06; C12N 15/82

(52) U.S. Cl. 800/288; 800/294; 435/419; 514/44; 435/325; 536/23.1

(57) ABSTRACT

The present invention provides plant-derived agents to interfere with the nonstructural NS1 gene from the influenza A virus subtype H1N1. More particularly, the siRNAs that exhibit strong inhibitory activity towards NS1, which effectively suppress replication of the influenza virus in mammalian cells. The invention further provides methods for production of siRNAs for the suppression of a broad range of influenza viral subtypes with sequence homologies.

This application claims priority benefit of U.S. Provisional Patent Application No. 60/673,100, filed on Apr. 20, 2005, which is incorporated herein by reference in its entirety.

Several publications are referenced herein by Arabic numerals within parentheses. Full citations for these references may be found at the end of the specification immediately preceding the claims. The entire contents of those publications are incorporated herein by reference.

### FIELD OF THE INVENTION

The present invention relates to the creation of transgenic tobacco plants that produce siRNAs targeted to the mRNA of the nonstructural protein NS1 from the influenza A virus subtype H1N1. The present invention further relates to plant-derived siRNAs to inhibit viral propagation through RNAi, and the application in control of viral-borne diseases.

### BACKGROUND OF THE INVENTION

RNA interference (RNAi) is an ancient and evolutionarily conserved activity in eukaryotes. It can result in RNA-mediated RNA degradation and/or translational repression in a sequence-specific manner. Originally described in plants as a concerted inactivation of host genes and transgenes transcribing the same or similar sequences [1], it has been confirmed to occur in many different organisms. Examples include quelling in Neurospora crassa [2], and RNAi in Caenorhabditis elegans [3], Drosophila [4] and mammals [5]. In all these cases, RNAi is achieved through several closely-coordinated steps: 1) an endonuclease dicer with RNase III activity cleaves the dsRNA into small interfering RNAs (siRNAs); 2) the siRNAs interact with a multicomponent nuclease to form an RNA-induced silencing complex (RISC); 3) the siRNA in the RISC directs the complex to the target RNA through sequence complementarity; 4) RNA polymerization begins from the siRNA to form dsRNA; and 5) the dsRNA is cleaved into siRNAs [6, 7]. The resulting siRNAs would then initiate additional RNA cleavage. In addition, siRNAs and related cellular microRNAs could also suppress target gene expression via translational repression [6].

Studies using synthetic [8], in vitro [9, 10] and in vivo transcribed [11, 12] siRNAs, as well as viral-mediated siRNA delivery [13], have demonstrated that well-designed siRNAs can effectively suppress target gene expression. Hence, RNAi technology could eventually be applied in the therapeutics of human and animal viral diseases where the molecular components (viral sequences) are known, and in the case of infectious diseases, of which the relevant pathogens have been identified. In plants, viral-resistance has already been achieved through a plant RNAi pathway termed post-transcriptional gene silencing (PTGS) [14].

Although some understanding of siRNA inhibition of viral propagation [8, 11] has been achieved, the local folding of the target RNAs that reduces siRNA accessibility within a transcript [15] makes it necessary to test out many different siRNAs before optimal transcript degradation can be attained [8, 16]. For example [8], 20 siRNA oligos were screened before identification of a few that could satisfactorily suppress replication of the influenza virus in mammalian cells. Also, siRNA-mediated gene suppression in mammals requires the dsRNA to be smaller than 30 bp to ensure specificity [17], as long dsRNA can provoke non-specific degradation of RNA transcripts and a general shutdown of protein translation [18]. Therefore, it is impossible to transfect mammalian cells with long-dsRNA-producing constructs essential for making multiple siRNAs. The high cost of RNA oligo synthesis and the toxic effects of long dsRNA in mammalian cells could be ameliorated by cost-effective techniques in simultaneous large-quantity production of different siRNAs to achieve a satisfactory level for RNAi-mediated gene suppression.

In the current invention, the inventors demonstrate that tobacco (Nicotiana tabacum cv. Samsun NN) can be engineered by Agrobacterium-mediated transformation to produce siRNAs targeting the mRNA for the nonstructural NS1 protein of the influenza virus A/WSN/33, subtype H1N1. The transgenic plants could effectively accumulate siRNAs that specifically target NS1 transcripts. Transfection of mammalian cells with plant-derived siRNAs followed by infection of the influenza virus revealed significant reduction in viral propagation. The present data demonstrate that plants can be used as an economical and sustainable source for large-scale production of diversified siRNAs.

### SUMMARY OF THE INVENTION

The invention provides a method of suppressing virus propagation in mammalian cells infected with the influenza virus comprising the steps of harvesting 5mNS1 siRNAs from the leaves of transgenic plants and transfecting confluent Madin-Darby canine kidney (MDCK) cells with 5mNS1 siRNAs.

The invention further provides a set of 5mNS1-derived siRNAs which binds to and deactivates mRNA of nonstructural protein NS1 from influenza A virus subtype H1N1. The invention also provides a “35S-s 5mNS1-TGA1 intron-as 5mNS1-nos” cassette, a vector comprising a plasmid and the foregoing cassette, and a cell or cell line made incorporating or transfected with that vector.

### BRIEF DESCRIPTION OF THE DRAWINGS

The above features and advantages of the present invention will be better understood with reference to the accompanying Detailed Description of the Preferred Embodiments, studied in conjunction with the accompanying drawings in which:

FIG. 1 shows the 5mNS1 sequence and the hairpin RNA construct used in producing 5mNS1 siRNAs in tobacco. [A] Schematic representation of NS1 mRNA. The cap and poly(A) tail structures are shown, and location of the 0.4-kb 5mNS1 fragment beginning from the first codon (atg) is indicated. [B] cDNA sequence of the 5mNS1, with the sequence of the synthetic siRNA NS-128 used by Ge et al. [8] underlined. [C] Diagram showing RNAi cassette in a binary vector. The sense (S) and antisense (AS) 5mNS1 fragments are separated by the Arabidopsis TGA1 intron, and is under the control of the CaMV 35S promoter.

FIG. 2 shows accumulation of the 5mNS1 siRNA in selected primary transformants and in wild-type tobacco (WT). [A] Twenty μg of total RNA from leaves of transgenic tobacco were separated on a 15% polyacrylamide gel containing 7 M urea, blotted and hybridized to ³²P-UTP labeled 5mNS1 riboprobes. [B] Normalization of RNA loading was based on the separation of 6 μg of total RNA on a 1.2% agarose gel.

FIG. 3 shows suppression of 5mNS1 transcript accumulation in siRNA-expressing lines. Wild-type tobacco and transgenic plants expressing different levels of 5mNS1 siRNAs were co-infiltrated with two binary vectors separately expressing 5mNS1 and a fusion fragment of EYFP-T2m. After two days, leaf samples were collected for RNA analysis by northern blot analysis. [A] Part of the T-DNA in the two binary vectors. [B] Northern blot analysis showing levels of EYFP-T2m and 5mNS1 transcripts in the different infiltrated samples. The 28S rRNA was stained with ethidium bromide. The 5mNS1 levels are lowered in transgenic plants when compared to levels in WT. [C] The 5mNS1 transcript level, as a percentage of the wild-type, was calculated with data from three separate infiltrations. The calculation was performed according to the formula of:

$$5 mNS 1 level ( \% of WT ) = \frac{\begin{matrix}( 100 ) \times ( 5 mNS 1 volume of transgenic line ) \times \\ ( EYFP volume of wild \text{-} type )\end{matrix}}{\begin{matrix}( 5 mNS 1 volume of wild type ) \times \\ ( EYFP volume of transgenic line )\end{matrix}}$$

FIG. 4 shows plant-derived 5mNS1 siRNAs can suppress replication of the influenza virus A/WSN/33 in mammalian cells. [A] Total RNA (10 μg) from primary transformants 1 and 2 was separated on a 15% polyacrylamide gel, blotted on to a Nylon membrane and probed with ³²P-UTP labeled 5mNS1 RNA probes. The quantity of siRNAs in the RNA samples was calculated based on its relative volume to that of known amount of synthetic siRNA oligo. These RNA preparations were then used for transfection of MDCK cells. [B] Normalization of RNA loading was based on the separation of 6 μg of total RNA on a 1.2% agarose gel. [C] Suppression of virus replication as revealed in 3 independent HA titre assays. MDCK cells were first transfected with water (mock), a siRNA oligo NS-128 used by Ge et al. [8], and RNA from wild-type tobacco (control) or from two transgenic lines (line 1 and line 2) expressing siRNAs, and were then infected by influenza virus strain A/WSN/33 24 hr post-transfection. HA titre was determined at 24 (blue boxes), 36 (red boxes) and 48 hr (yellow boxes) post-infection.

### DETAILED DESCRIPTION OF THE PREFERRED EMBODIMENTS

The invention provides a method of producing an agent comprising the steps of: (a) amplifying a region of the H1N1 NS1 gene by PCR; (b) cloning the PCR product in pGEM-T vector; (c) digesting the pGEM-T Easy derivative with restriction enzymes; (d) cloning the resulting fragments into corresponding sites in pBluescript SKII(−) derivatives to form a “35S-s 5mNS1-TGA1 intron-as 5mNS1-nos” cassette; (e) digesting the cassette with restriction enzymes; (f) cloning of the digested cassette into the T-DNA in a pBI101 backbone plasmid derivative; and (g) mobilizing the binary vector into Agrobacterium tumefaciens strain GV3101/MP90 for transformation of tobacco cultivar Samsun NN. The agent may be small interfering RNA or siRNA, and the PCR may be performed by using two specific primers. The primers are represented by one of the following sequences: i.) SEQ ID 1: 5′-gggcggccgcggatccatggacccaaacactgtg-3′; or ii.) SEQ ID 2: 5′-caactagtatttcgctttcagtatga-3′. The restriction enzymes may be BamHI, SpeI, NotI, and KpnI.

The invention also provides a method of suppressing virus propagation in mammalian cells infected with the influenza virus, comprising the steps of: (a) harvesting 5mNS1 siRNAs from the leaves of transgenic plants; and (b) transfecting confluent Madin-Darby canine kidney (MDCK) cells with plant-derived 5mNS1 siRNAs. Preferably, the influenza virus is H1N1, and the plant-derived 5mNS1 siRNAs have anti-viral efficacy. The invention further provides a set of 5mNS1 siRNA which bind to and deactivate mRNA of nonstructural protein NS1 from influenza A virus subtype H1N1, and a “35S-s 5mNS1-TGA1 intron-as 5mNS1-nos” cassette. In another embodiment, the invention provides a vector comprising a plasmid and the cassette and a cell or cell line incorporating or transfected with the plasmid and the cassette.

### EXPERIMENTAL SECTION

The following experimental section is intended to be illustrative, and not limiting of the scope of the invention. Described herein are plant-derived siRNAs against the influenza virus could inhibit viral replication. The siRNAs are derived from a 0.4-kb fragment representing the 5′-portion of the NS1 gene in strain A/WSN/33, subtype H1N1. This invention also provides cost-effective technique in utilizing transgenic plants for large-scale siRNA production.

Influenza A viruses are medically-important viral pathogens that cause significant mortality and morbidity throughout the world. Their easy transmission, antigenic shift and drift have made current methodology of vaccination and therapy limited in efficacy [22]. Inhibitors of the anti-M2 ion channel and neuraminidase are common drugs for influenza, but both have their drawbacks. The anti-M2 ion channel inhibitors (e.g., amantidine) induce viruses to develop drug-resistant mutations, while the neuraminidase inhibitors (e.g., Tamiflu), though very potent, are effective only at early disease onset. To investigate if plant-derived siRNAs against the influenza virus could inhibit viral replication, the inventors selected a 0.4-kb fragment representing the 5′-portion of the NS1 gene in strain A/WSN/33, subtype H1N1. The NS virion RNA (vRNA) consists of about 890 nucleotides and encodes two nonstructural proteins, NS1 and NS2. The sequence of this vRNA is highly-conserved among different subtypes of influenza viruses [23]. The NS1 protein has not only been proposed to regulate viral replication cycle, splicing and translation of mRNAs [24], but also been shown to have inhibitory effect on cellular mRNA maturation and cellular antiviral response [25]. Thus, the NS1 gene plays an important role in virus replication and virus-host interactions.

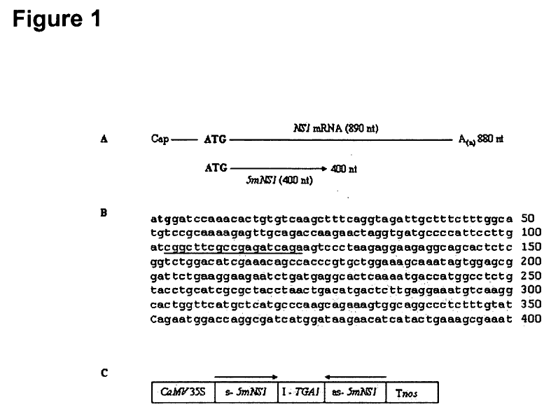

The chosen 0.4-kb fragment was amplified by Polymerase Chain Reaction (PCR) using forward primer 5′-gggcggccgcggatccatggacccaaacactgtg-3′with NotI (in italics) and BamHI (in bold) sites incorporated at its 5′-end, and reverse primer 5′-caactagtatttcqctttcaqtatga-3′with an added SpeI site (in italics). The underlined nucleotides represent NS1 sequences. The PCR product was initially cloned in pGEM-T Easy vector (Promega) for verification of DNA sequence. Subsequently, the PGEM-T Easy derivative was digested with either BamHI and SpeI or NotI and SpeI. The 0.4-kb BamHI-SpeI 5mNS1 fragment was cloned into corresponding sites in a pBluescript SKII(−) derivative that contains the Arabidopsis TGA1 intron [19] inserted at its SpeI-XbaI site. Next, the 0.4-kb NotI-SpeI 5mNS1 fragment from the pGEM-T Easy derivative was cloned in the NotI-XbaI site of the pBluescript SK(II)(−) derivative containing the DNA fusion of “sense 5mNS1-TGA1 intron,” to generate a dsRNA cassette “sense (s) 5mNS1-TGA1 intron-antisense (as) 5mNS1.” This cassette was then released by NotI and BamHI digestion, and, with the help of a NotI/XbaI adaptor (upper strand, 5′GGCCGAGTTGTTA3′; lower strand, 5′CTAGTAACAACTC3′), was cloned in the BamHI-XbaI site between the CaMV 35S promoter and the nos terminator, in another pBluescript SKII (−) derivative. The resulting vector therefore contains a cassette of “35S-s 5mNS1-TGA1 intron-as 5mNS1-nos”(FIG. 1C). This cassette was further digested with NotI and KpnI, and was cloned into corresponding sites within the T-DNA in a pBI101 backbone plasmid derivative (Clontech, Palo-Alto, USA). The binary vector was then mobilized into Agrobacterium tumefaciens strain GV3101/MP90 for transformation of tobacco cultivar Samsun NN by the leaf-disk procedure [20]. Hence, transgenic plants obtained in Agrobacterium-mediated plant transformation from this binary vector should produce hairpin dsRNA, which would subsequently be processed into siRNAs by the PTGS machinery.

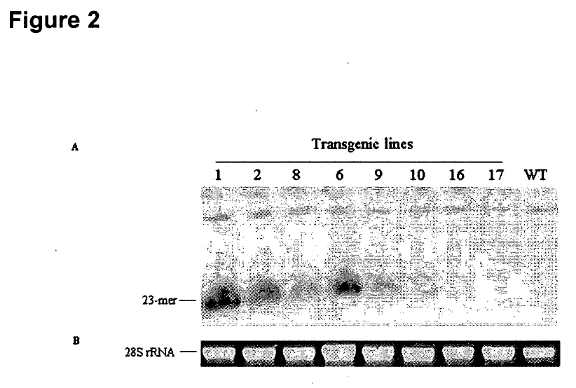

Reports have shown that “sense-antisense” cassettes can be transcribed to produce siRNAs after transfection of host cells [11, 26, 27]. To investigate if the construct generated in this study (FIG. 1C) could produce siRNA in transgenic tobacco, RNAs were extracted from tobacco leaves using TRIzol (Invitrogen). Twenty μg of total RNA were separated on a 15% polyacrylamide gel containing 7 M urea, and were electroblotted onto a nitrocellulose membrane (GeneScreen Plus®, PerkinElmer Life Sciences, Inc.). The blot was then hybridized overnight at 42° C. to ³²P-UTP-labeled 5mNS1 riboprobes generated using the Riboprobe® in vitro Transcription Systems (Promega), in a solution of 50% (v/v) formamide, 250 mM NaCl, 7% SDS and 125 mM phosphate buffer, pH 7.0. After hybridization, the blot was washed twice with 2×SSC plus 0.5% SDS, and was then analyzed using a phospho-imager. The volumes of the synthetic siRNA and of the siRNA from transgenic plants were measured using an ImageQuant software (Molecular Dynamics), and the amount of siRNA in the plant RNA sample was calculated based on its volume relative to that of synthetic, known amount of RNA oligos. Of the 21 independent transformants screened, 13 showed obvious siRNA production. The levels of siRNA accumulation in different lines varied, some produced obvious signals after an overnight exposure using a phospho-imager, while others barely yielded visible signals (data not shown). The siRNA signals in selected transgenic lines are shown in FIG. 2. Transgenic lines 1 and 2 had apparent accumulation of 5mNS1 siRNAs, while lines 8 and 9 produced much lower levels of the same siRNAs. In line 10, the siRNAs were barely detectable.

A variation in siRNA levels may be due to several reasons. First, T-DNA location in the genome could affect expression. In Agrobacterium-mediated plant transformation, T-DNA is transferred from the bacterium to the eukaryotic host cell and further integrated into the host genome [28]. If the transgenes were inserted in the genome where active transcription occurs, the transgene would be active. Otherwise, it would be less active or even silent. Second, the copy number of the transgene may be a contributing factor in expression levels, although in some cases, transgene activity may not be directly proportional to its copy number due to co-suppression. Third, methylation of transgene may occur, especially at or near the promoter, if it is considered foreign. As a safeguard, the host generally has a mechanism to methylate and inactivate the transgene. This has been reported with foreign DNA expressing dsRNA in PTGS [29, 30], and is supported by a requirement of DNA methylase in initiating RNA-dependent DNA methylation [31].

As revealed by an increasing number of reports, siRNA is the hallmark in triggering RNAi. Therefore, the accumulated 5mNS1 siRNAs in the transgenic plants should initiate degradation of NS1 transcripts or endogenous tobacco transcripts with sequences complementary to 5mNS1. A BLAST analysis was performed with 5mNS1 as query sequence for such complementation in transcripts of tobacco or species evolutionarily close to tobacco, but no match was identified. Northern blot analysis of tobacco total RNA with the 5mNS1 probe also did not yield any obvious bands. Therefore, 5mNS1 does not seem to share homology to any tobacco transcripts, and would not cause unintended degradation of RNA transcribed from endogenous genes. This is consistent with the fact that no abnormal phenotypes were observed in all the transgenic lines (data not shown).

To test if the plant-derived 5mNS1 siRNAs were functional in degrading NS1 transcripts specifically, 5mNS1- and EYFP-T2m-expressing binary vectors were introduced into Agrobacterium cells by inoculating in an induction solution containing 1 g/l NH₄Cl, 0.3 g/l MgSO₄.7H₂O, 0.15 g/l KCl, 0.01 g/l CaCl₂, 0.0025 g/l FeSO₄.7H₂O, 2 mM phosphate, 1% glucose, 20 mM 2-(N-morpholino)ethanesulfonic acid (MES, pH5.5), 100 μM acetosyringone, 50μg/ml kanamycin and 50 μg/ml gentamycin. The EYFP-T2m contains EYFP fused in-frame to a mutant version of the Arabidopsis TGA2 gene (T2m), and is used as an expression reference after agroinfiltration [21]. Following overnight culture at 28° C., the cells were collected by centrifugation at 3,000 ×g for 15 min, and then resuspended in an infiltration solution containing 10 mM MES (pH5.5), 10 mM MgSO₄ and 100 μM acetosyringone. The resuspended Agrobacterium cells were adjusted to an OD₆₀₀ of 0.8 with the same solution before infiltration of tobacco leaves using a 1-ml syringe. After two days, total RNA was extracted from the infiltrated leaf areas for northern blot analysis.

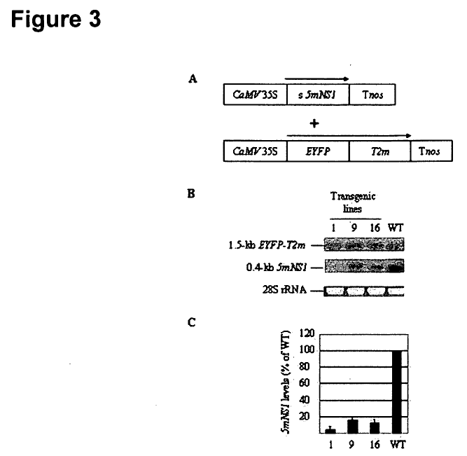

Five μg of total RNA, extracted from the agroinfiltrated and non-infiltrated leaf areas, were separated on a 1.2% agarose gel, blotted with 20×SSC onto a nitrocellulose membrane, and hybridized to ³²P-dCTP labeled DNA probes generated from 5mNS1 and EYFP DNA fragments using a Rediprime™II Random Prime Labelling System (Amersham, UK). Hybridization was performed at 65° C. overnight in a buffer containing 250 mM NaCl, 7% SDS and 125 mM phosphate, pH 7.0. After hybridization, the blot was washed twice at room temperature in 2×SSC plus 0.5% SDS, then at 65° C. for 15 min in 0.2×SSC plus 0.1% SDS. The blot was analyzed using a phospho-imager. As shown in FIG. 3B, all the 3 transgenic lines 1, 9 and 16, representing high, middle and low accumulation of 5mNS1 siRNA, respectively, had reduced 5mNS1 RNA levels, indicating that plant-derived 5mNS1 siRNAs indeed triggered PTGS of NS1 in vivo. A negative correlation was observed between the levels of 5mNS1 siRNAs and 5mNS1 transcripts in infiltrated tobacco leaves. To obtain a percentage of the 5mNS1 transcript level in the transgenic lines relative to that of wild-type, volumes of each 5mNS1 band and of the reference EYFP band were determined using the ImageQuant software, and percentage was calculated using the formula described in the FIG. 3 legend. In transgenic tobacco line 1, which had the highest level of 5mNS1 siRNA accumulation, the percentage was only 4.7±3.0%, demonstrating high efficiency of this line in 5mNS1-specific RNA degradation (FIG. 3C).

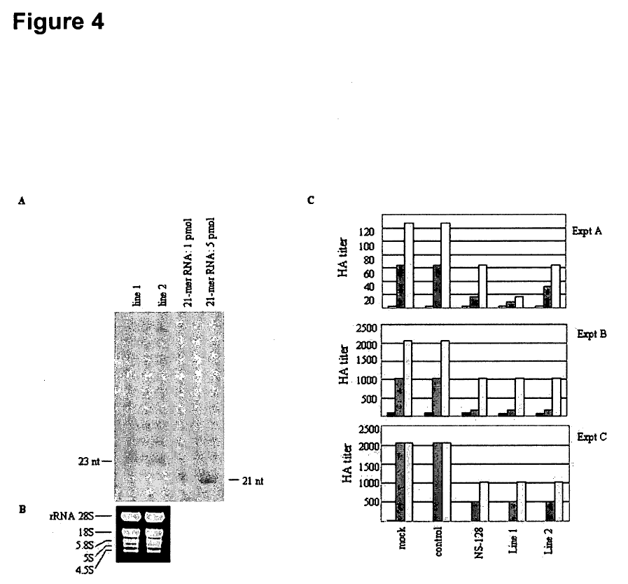

In RNAi studies, synthetic or in vitro expressed siRNAs have been used in transfection of target cells [26], and injection of worms [32] and animals [33], for evaluation of siRNA efficacy. To test if the 5mNS1 siRNAs produced in transgenic tobacco could be potentially used in suppressing viral propagation in mammalian cells infected with the influenza virus, 5mNS1 siRNAs were harvested from the leaves of transgenic plants for transfection of MDCK cells. Confluent Madin-Darby canine kidney (MDCK) cells grown in a T-175 flask were washed twice with phosphate-buffered saline (PBS) and trypsinized for 10 min in 10 ml trypsin solution at 37° C. After termination of trypsinization with 20 ml PBS, the cells were collected by centrifugation for 5 min at 1,500 rpm with an Eppendorf 5810R centrifuge, and were washed twice in 30 ml cold PBS, followed by one wash in 30 ml cold RPMI1640 medium (GIBCO), before resuspension in cold RPMI1640 to a density of 1×10⁷ cells/ml. The amount of siRNA in total RNA was quantified by siRNA analysis (FIG. 4A). The siRNA oligo NS-128, (5′-CGGCUUCGCCGAGAUCAGAdAdT-3′) was used as a positive control, since it has been proven best of three NS1 -targeting siRNA oligos [8].

Cells transfected with RNA from non-transformed plants were the negative control, and cells transfected with water constituted the mock transfection. Subsequently, 500 μl resuspended cells were transferred into a 0.4-cm pre-chilled cuvette, and were mixed with 10 μl water, 10 μl water with 42 ng NS-128, 10 μl wild-type RNA sample or 10 μl RNA sample containing 42 ng siRNAs from transgenic plant. Equal amounts of total RNA from wild-type or transgenic plants were used. The cuvette was kept on ice for 10 min, before electroporation at 0.4 kV and 960 μF using a gene pulser system (Bio-Rad). Cells were then transferred into 5.6 ml of pre-warmed MDCK medium (MEM, 10% cow serum, 1% penicillin and 1% streptomycin). Three ml were transferred into a 6-well plate and incubated at 37° C. for 24 hr before infection with the influenza virus.

Twenty four hr after transfection, cells in each well were washed twice with PBS, and 300 μl diluted influenza virus strain A/WSN/33 (MOI=0.001 in PBS) was added into the well. After shaking the mixture for 1 hr, the viruses in the supernatant were discarded, and 2 ml infection medium (0.5 μ/ml TPCK-trypsin (Sigma), 0.5% FCS (Gibco), 1% PS with MEM (Gibco)) were added into the well. The cells were then incubated at 37° C. Supernatants were collected at different post-infection time points for the HA titre test as described [8]. The HA titre, which is an indicator of virus replication, was determined at 24, 36 and 48 hr post-infection. The mock-transfected and the negative control cells showed similar HA titre, indicating that RNA from wild-type tobacco plants did not suppress viral replication. Though the HA titre values varied in 3 separate sets of transfection and infection studies, a phenomenon unavoidably associated with conditions of the cells e.g. passage history, both plant-derived and synthetic siRNAs significantly reduced H1N1 viral replication. The antiviral effect of siRNA was most prominent at 36 hr post-infection (FIG. 4C). In one set of experiments, plant-derived siRNA proved superior to the NS-128 oligo (FIG. 4C, experiment A).

These results strongly support the conclusion that 5mNS1 siRNA from transgenic plants can effectively suppress replication of the influenza virus in mammalian cells. In addition, plant siRNAs showed similar suppression ability as the synthetic siRNA NS-128, demonstrating that plant-derived siRNAs confer the same efficacy. Given the fact that transgenic plants can generate siRNAs targeting different areas of the 5mNS1 transcript, and that NS1 sequences are highly-conserved among influenza viruses [23], 5mNS1 siRNAs from transgenic plants should suppress the replication of a broad range of influenza viral subtypes with sequences homologous to the 5mNS1.

While these results clearly indicate antiviral effects of plant-derived 5mNS1 siRNAs, the invention is primarily focusing on developing a strategy for economical and sustainable production of siRNAs. Besides using transgenic technology described herein, a pool of siRNAs can also be generated with Dicer-dependent kits. When compared with the transgenic approach, the later method is much more expensive, since it involves more expensive reagents (i.e. NTPs, Dicer, RNA polymerase), more complicated steps (i.e. in-vitro transcription, in-vitro cleavage of dsRNA, clean-up) and a more experienced technician/scientist. The high cost not only limits production scale, but also requires repetition of the production process if the siRNAs are to be used over and over again. Therefore, the current proof-of-concept invention demonstrates that transgenic plants are superior to the commercial kits for siRNA production, and the time taken for generating them would be well compensated.

Though the NS1-targeting siRNAs possesses anti-viral effects, those against the NP genes may be more potent in suppressing viral replication, as suggested by studies using mammalian cells [8] and animals [34]. In both studies, one of the NP-targeting siRNAs, NP-1496, significantly reduced the virus titres. These observations indicate that mRNA of the NP gene might be a better target of siRNA, if positional effects on siRNA accessibility could be faithfully addressed. In our future study of using transgenic plant-derived siRNAs for viral suppression, generating NP-targeting siRNAs or a combination of siRNAs targeting multiple viral components may be a more effective practice.

In conclusion, 5mNS1 siRNAs capable of activating RNAi in mammalian cells against NS1 were produced in transgenic tobacco plants. The efficacy of the plant-derived siRNAs was tested in vivo by agroinfiltration of the 5mNS1-expressing construct in leaves of transgenic tobacco, and in vitro by application of these siRNAs in mammalian cells to inhibit influenza virus replication. This cost-effective technique in utilising transgenic plants for large-scale siRNA production could have advantages over current methods involving the use of synthetic RNA oligos, the expression of short hairpin RNA in E. coil [35, 36] and the transfection of mammalian cells with short dsRNA. In addition, plant cells can apparently tolerate expression of long dsRNAs, enabling the length of the target gene fragment to be easily manipulated for optimal suppression. Moreover, fragments producing siRNAs targeting multiple sites of the viral genome can be fused together so that one transgenic plant can produce siRNAs for simultaneous silencing of multiple genes. This could provide a more robust and sustained viral protection minimizing the likelihood of the virus developing resistance to the siRNA through mutation of the target sequence.

### REFERENCES

[1] Napoli, C. et al., Plant Cell, 2, 279-289 (1990).
[2] Cogoni, C. and Macino, G., Proc. Natl. Acad. Sci. USA, 94,10233-10238 (1990).
[3] Fire, A., et al., Nature, 391, 806-811 (1998).
[4] Zamore, P. D., et al., Cell, 101, 25-33 (2000).
[5] Wianny, F. and Zernicka-Goetz, M., Nat. Cell Biol., 2, 70-75 (2000).
[6] Meister, G. and Tuschl, T., Nature 431, 343-349 (2004).
[7] Denli, A. M. and Hannon, G. J., Trends Biochem. Sci. 28, 196-201 (2003).
[8] Ge, Q., et al., Proc. Natl. Acad. Sci. USA, 100, 2718-2723 (2003).
[9] Sohail, M., et al., Nucleic Acids Res., 31,e38 (2003).
[10] Myers, J. W., et al. Nat. Biotechnol. 21, 324-328 (2003).
[11] Lee, N. S., et al., Nat. Biotechnol. 19, 500-505 (2002).
[12] Kaykas, A. and Moon, R. T., BMC Cell Biol. 5, 16-26 (2004).
[13] Xia, H. B., et al., Nat. Biotechnol. 20, 1006-1010 (2002).
[14] Tenllado, F., et al., Virus Res. 102, 85-96 (2004).
[15] McManus, M. T. and Sharp, P. A., Nat. Rev. Genet. 3, 737-747 (2002).
[16] Holen, T., et al. Nucleic Acids Res. 30,1757-1766 (2002).
[17] Gil, J. and Esteban, M., Apoptosis 5, 107-114 (2000).
[18] Stark, G. R., et al., Annu. Rev. Biochem. 67, 264-277 (1998).
[19] Kotani, H., et al., DNA Res. 5, 203-216 (1998).
[20] Gallois P. and Marinho P., Methods Mol Biol 49, 3948 (1995).
[21] Pontier, D., et al., Plant J. 27, 529-538 (2001).
[22] Webby, R. J. and Webster, R. G., Philos. Trans. R. Soc. London 356, 1817-1828 (2001).
[23] Suarez, D. L. and Perdue, M. L., Virus Res. 54, 59-69 (1998).
[24] Garcia-Sastre, A., et al., Virology 252, 324-330 (1998).

[25] Krug R. M., et al., Virology 309, 181-189 (2003).
[26] Sui, G. C., et al., Proc. Natl. Acad. Sci. USA 99, 5515-5520 (2002).
[27] Klahre, U., et al., Proc. Natl. Acad. Sci. USA 99, 11981-11986 (2002).
[28] Tzfira T., et al., Trends Genet. 20, 375-383 (2004).
[29] Chan, S. W. L., et al., Science 303, 1336 (2004).
[30] Aufsatz, W., et al., Proc. Natl. Acad. Sci. USA 99 Supp 14, 16499-16506 (2002).
[31] Cao, X., et al., Curr. Biol. 13, 2212-2217 (2003).
[32] Newmark, P. A., et al., Proc. Natl. Acad. Sci. USA 100 Supp 11, 11861-11865 (2003).
[33] Sorensen, D. R., et al., J. Mol. Biol. 327, 761-766 (2003).
[34] Tompkins S. M., et al., Proc. Natl. Acad. Sci. USA 101,8682-8686 (2004).
[35] Timmons, L. and Fire, A., Nature 395, 854 (1998).
[36] Ketting, R. F., et al., Genes Dev. 15, 2654-2659 (2001).

What is claimed is:

1. A method of producing small interfering RNA (siRNA) comprising the steps of:
a.) amplifying a region of the H1N1NS1 gene by PCR;
b.) cloning the PCR product in pGEM-T vector;
c.) digesting the PGEM-T Easy derivative with restriction enzymes;
d.) cloning the resulting fragments into corresponding sites in pBluescript SKII(−) derivatives to form a 35S-s 5mNS1-TGA1 intron-as 5mNS1-nos cassette;
e.) digesting the cassette with restriction enzymes;
f.) cloning the digested cassette into the T-DNA in a pBI101 backbone plasmid derivative; and
g.) mobilizing the binary vector into Agrobacterium tumefaciens strain GV3101/MP90 for transformation of tobacco cultivar Samsun NN.

2. The method of claim 1, wherein the PCR is performed by using two specific primers.

3. The method of claim 2, wherein the primers are represented by one of the following sequences:
a.) SEQ ID 1:
5′-gggcggccgcggatccatggacccaaacactgtg-3′;
or
b.) SEQ ID 2:
5′-caactagtatttcgctttcagtatga-3′.

4. The method of claim 1, wherein the restriction enzymes are BamHI, SpeI, NotI, and KpnI.

5. A method of suppressing viral propagation in mammalian cells infected with the influenza virus comprising the steps of:
producing transgenic plants transfected with a vector including a 35s-s5mNS1-TGA1 intron-as 5mNS1-nos cassette;
harvesting 5mNS1 siRNAs from the leaves of transgenic plants; and
transfecting mammalian cells with siRNA.

6. The method of claim 5, wherein the agent is influenza virus.

7. The method of claim 5, wherein the 5mNS1 siRNA has anti-viral efficacy.

8. A 5mNS1 siRNA which binds to and deactivates mRNA of nonstructural protein NS1 from influenza A virus subtype H1N1.

9. A 35S-s 5mNS1-TGA1 intron-as 5mNS1-nos cassette.

10. A vector comprising a plasmid and the cassette of claim 9.

11. A cell or cell line made in accordance with the method of claim 5.

12. A cell or cell line made in accordance with the method of claim 7.
